(19) United States
(12) Patent Application Publication
(10) Pub. No.: US 2011/0091402 A1
(43) Pub. Date: Apr. 21, 2011

(54) ANTIPERSPIRANT PRODUCTS AND PROCESSES FOR FABRICATING THE SAME

(75) Inventors: Thomas Doering, Scottsdale, AZ (US); Cheryl Galante, Scottsdale, AZ (US)

(73) Assignee: The Dial Corporation, Scottsdale, AZ (US)

(21) Appl. No.: 12/581,516

(22) Filed: Oct. 19, 2009

(51) Int. Cl. A61K 8/58; A61Q 15/00

(52) U.S. Cl. 424/65

(57) ABSTRACT

Antiperspirant products and processes for making them are provided. In one embodiment, an antiperspirant product comprises an active antiperspirant compound and a carrier having a first volatility rate in an air ambient at about 50° C. and 1 atm after a first time period and a second volatility rate in an air ambient at about 50° C. and 1 atm after a second time period, wherein the second volatility rate is less than the first volatility rate and the first time period is less than the second time period.

### FIELD OF THE INVENTION

The present invention generally relates to antiperspirant products and processes for making them, and more particularly relates to antiperspirant products that exhibit reduced white residue and processes for making them.

### BACKGROUND OF THE INVENTION

Antiperspirants are popular personal care products used to prevent or eliminate sweat and body odor caused by sweat. Antiperspirant sticks are desired by a large majority of the population because of the presence of active antiperspirant compounds that minimize or prevent the secretion of sweat by blocking or plugging ducts of sweat-secreting glands, such as those located at the underarms. Antiperspirants typically comprise the active antiperspirant compound in a carrier that permits the antiperspirant product to be applied to the skin by swiping or rubbing the stick across the skin, typically of the underarm. Upon application, the carrier evaporates, releasing the active antiperspirant compound from the antiperspirant product to form plugs in the sweat ducts.

However, antiperspirant users often are disappointed in the chalky, white residue that remains on the skin after the carrier evaporates. The white residue is formed from the active antiperspirant compound and waxes. Conventional antiperspirant sticks typically comprise a cyclomethicone, such as cyclopentasiloxane or cyclotetrasiloxane, as the carrier. As illustrated in FIG. 1, cyclomethicones such as cyclopentasiloxane have a substantially constant volatility or evaporation rate (herein referred to as volatility rate), similar to that of water. The longer the antiperspirant is on the skin, the more the carrier evaporates and the more white residue is left behind on the skin.

Accordingly, it is desirable to provide antiperspirant products that exhibit reduced white residue compared to conventional antiperspirant products. In addition, it is desirable to provide antiperspirant products that comprise a carrier having a varying volatility rate. It is also desirable to provide processes for making the antiperspirant products. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Antiperspirant products and processes for making them are provided. In accordance with an exemplary embodiment, an antiperspirant product comprises an active antiperspirant compound and a carrier having a first volatility rate in an air ambient at about 50° C. and 1 atm after a first time period and a second volatility rate in an air ambient at about 50° C. and 1 atm after a second time period, wherein the second volatility rate is less than the first volatility rate and the first time period is less than the second time period.

In accordance with another exemplary embodiment, an antiperspirant product comprises an active antiperspirant compound and a carrier comprising a material selected from the group consisting of polydimethyl siloxane, isoparaffin/alkane compounds, and combinations thereof.

In accordance with a further exemplary embodiment, a process for making an antiperspirant product comprises providing a carrier having a first volatility rate after about 10 minutes in an air ambient at about 50° C. and 1 atm and a second volatility rate after about 100 minutes in an air ambient at about 50° C. and 1 atm, wherein the second volatility rate is less than the first volatility rate. The carrier and an active antiperspirant compound are combined at a first temperature to form a mixture and the mixture is cooled to a lower second temperature.

### BRIEF DESCRIPTION OF THE DRAWINGS

The present invention will hereinafter be described in conjunction with the following drawing figures, wherein like numerals denote like elements, and wherein:

FIG. 1 is a graph of the volatility (percent weight lost) of cyclopentasiloxane over time at 50° C. and at 1 atmosphere (atm); and

FIG. 2 is a graph of the volatility (percent weight lost) of polydimethyl siloxane and SiClone Sr-5 over time at 50° C. and at 1 atm compared to that of cyclopentasiloxane.

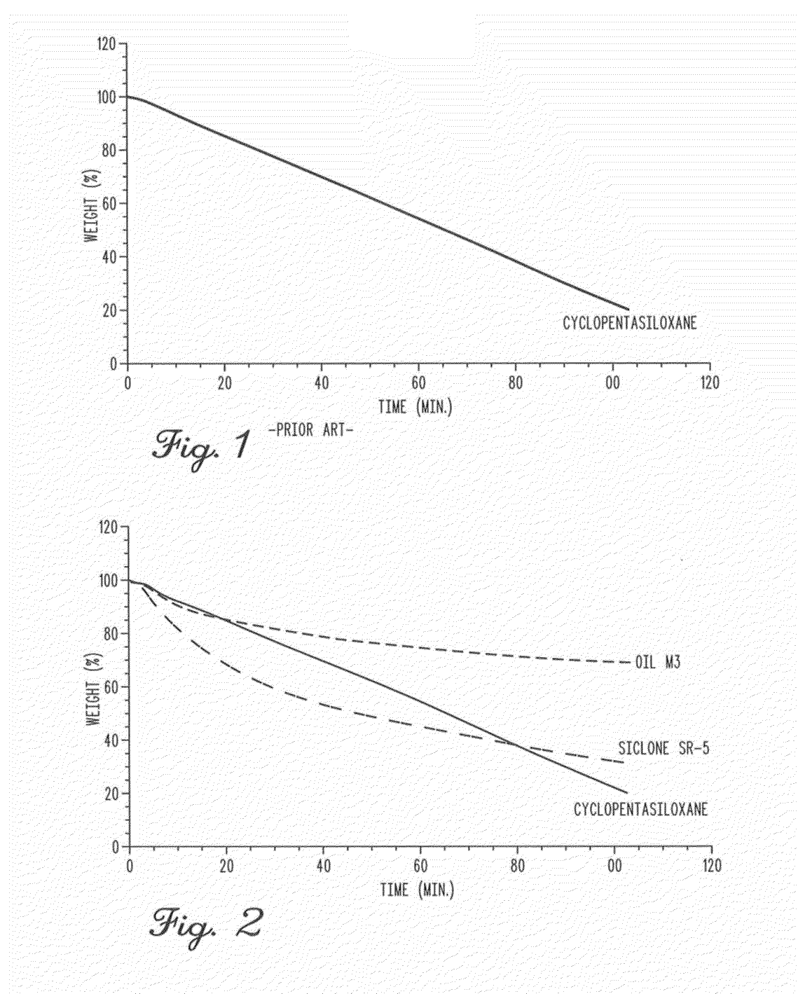

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to antiperspirant products that exhibit reduced white residue deposition over time compared to conventional antiperspirant products, in particular conventional antiperspirant products comprising only a cyclomethicone as a carrier. Specifically, the various embodiments of the antiperspirant products contemplated herein comprise an active antiperspirant compound and a carrier having a first volatility rate after a first time period and a second volatility rate after a second, longer time period, wherein the second volatility rate is less than the first volatility rate. In this regard, within the first few minutes of application of the antiperspirant product to the skin, the carrier evaporates quickly, leaving behind the active antiperspirant compound to plug sweat ducts of sweat-producing glands in the skin. After the antiperspirant product has been on the skin for an amount of time, the carrier evaporates less quickly, thus remaining on the skin to help mask white residue.

In this regard, in one exemplary embodiment of the present invention, the antiperspirant product comprises a carrier having a first volatility rate in an air ambient at about 50° C. and 1 atm. after a first time period and a second volatility rate in an air ambient at about 50° C. and 1 atm. after a second time period, wherein the second volatility rate is less than the first volatility rate and the first time period is less than the second time period. In a preferred embodiment, the carrier has a volatility rate in the range of about more than 7% weight loss after about 10 minutes in an air ambient at about 50° C. and 1 atm and a volatility rate in the range of about less than 75% weight loss after about 100 minutes in an air ambient at about 50° C. and 1 atm. In a more preferred embodiment, the carrier has a volatility rate in the range of about 10 to about 30% weight loss after about 10 minutes in an air ambient at about 50° C. and 1 atm and a volatility rate in the range of about 20 to about 70% weight loss after about 100 minutes in an air ambient at about 50° C. and 1 atm. As used herein, the term “volatility rate” refers to the percentage of weight of the carrier lost due to evaporation over a given time period. The volatility rates of the carrier can be measured using the following testing procedure. Twenty (20) micron liters of carrier is loaded into a differential scanning calorimetry (DSC) cell of about 6 millimeters (mm) in diameter and about 1 mm in depth. The DSC cell sits on a thermogravimetric analysis pan at 1 atm. The temperature of the carrier is ramped up to about 50° C. at a speed of about 10° C./minute and the temperature then is maintained at about 50° C. for 100 minutes. Once the temperature reaches 50° C., the weight of the carrier is measured every 30 seconds. The volatility rates are calculated by measuring the amount of weight lost by the carrier at 10 minutes after reaching 50° C. and at 100 minutes after reaching 50° C. and dividing each value of the weight lost by the total initial weight of the carrier and multiplying by 100.

Examples of carriers suitable for use in the various embodiments of the antiperspirant products contemplated herein include those comprising polydimethyl siloxane, such as for example, Oil M 3, available from Momentive Performance Materials of Albany, N.Y., an isoparaffin/alkane compound, such as SiClone SR-5 (C13-C16 isoparaffin/C12-C14 isoparaffin/C13-C15 alkane) available from Presperse LLC of Somerset, N.J., or the like, or combinations thereof. FIG. 2 is a graph of the volatility rates of Oil M 3 and SiClone SR-5 compared to the volatility rate of cyclopentasiloxane. As is evident from the graph, while cyclopentasiloxane has a constant volatility rate over time, both Oil M 3 and SiClone SR-5 exhibit a higher volatility rate within the first 10 minutes of exposure to air ambient at 50° C. and 1 atm and a lower volatility rate after about 100 minutes of exposure to air ambient at 50° C. and 1 atm. The carrier may comprise about 100% of polydimethyl siloxane or about 100% isoparaffin/alkane compound or may comprise one or both of these materials along with other carrier agents. For example, in one embodiment, the carrier may comprise a 20/80 vol. %, 40/60 vol. %, 50/50 vol. %, 60/40 vol. %, or 80/20 vol. % mixture of a polydimethyl siloxane and an isoparaffin/alkane compound. In another embodiment, the carrier may comprise a 20/80 volume percent (vol. %), 40/60 vol. %, or 50/50 vol. % mixture of a cyclomethicone, such as cyclopentasiloxane, cyclotetrasiloxane, or the like, and polydimethyl siloxane, respectively. In another embodiment, the carrier may comprise a 20/80 vol. % or 40/60 vol. % mixture of a cyclomethicone and an isoparaffin/alkane compound, respectively. In yet another embodiment, the carrier may comprise a 20/50/30 vol. % mixture of a cyclomethicone, polydimethyl siloxane, and isoparaffin/alkane. Of course, any other combination of these or other carrier agents that meet the above-identified specifications can be used in the various embodiments of the antiperspirant products contemplated herein. In a preferred embodiment, the carrier comprises polydimethyl siloxane. In another preferred embodiment, the carrier comprises about 30 to about 60 wt. % of the antiperspirant product. In a more preferred embodiment, the antiperspirant product comprises about 40 wt. % polydimethyl siloxane.

The various embodiments of the antiperspirant products also comprise a water-soluble active antiperspirant compound. Active antiperspirant compounds contain at least one active ingredient, typically metal salts, that are thought to reduce sweating by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so sweat cannot diffuse to the skin surface. Some active antiperspirant compounds that may be used in the antiperspirant product include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b} x (H₂O), wherein a is from 2 to about 5; a and b total to about 6; x is from 1 to about 6; and wherein a, b, and x may have non-integer values. Exemplary zirconium salts include those having the general formula ZrO(OH)₂₋ₐClₐ x (H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the active antiperspirant compound is aluminum zirconium pentachlorohydrex glycine. In a more preferred embodiment, the antiperspirant product comprises an active antiperspirant compound at an active level of about 8 to about 30 wt. % of the total antiperspirant product. In a most preferred embodiment, the antiperspirant product comprises about 21-22 wt. % aluminum zirconium pentachlorohydrex glycine.

Further included in the antiperspirant product is at least one structurant and/or gellant (hereinafter referred collectively as “structurant”) that facilitates the solid consistency of the antiperspirant stick product. Naturally-occurring or synthetic waxy materials or combinations thereof can be used as such structurants. Suitable structurants, including waxes and gellants, are often selected from fatty alcohols often containing from 12 to 30 carbons, such as stearyl alcohol, behenyl alcohol and sterols such as lanosterol. As used herein, the term “fatty” means a long chain aliphatic group, such as at least 8 or 12 linear carbons, which is frequently not branched (linear) and is typically saturated, but which can alternatively be branched and/or unsaturated. It is possible for the fatty acid to contain a hydroxyl group, as in 12-hydroxystearic acid, for example as part of a gellant combination, and to employ amido or ester derivates thereof.

Other structurants can comprise hydrocarbon waxes such as paraffin waxes, microcrystalline waxes, ceresin, squalene, and polyethylene waxes. Other suitable structurants are waxes derived or obtained from plants or animals such as hydrogenated castor oil, hydrogenated soybean oil, carnabau, spermacetti, candelilla, beeswax, modified beeswaxes, and Montan wax and individual waxy components thereof. It is especially suitable herein to employ a mixture of wax structurants. Suitable mixtures of structurants can reduce the visibility of active antiperspirant compounds deposited on the skin and result in either a soft solid or a firm solid. In an exemplary embodiment, the surfactant(s) comprise about 10 to about 35 wt. % of the total antiperspirant product. In a preferred embodiment, the antiperspirant product comprises a mixture of stearyl alcohol and hydrogenated castor oil. In a more preferred embodiment, the antiperspirant product comprises about 12 to about 25 wt. % stearyl alcohol and about 1.5 to about 7 wt. % hydrogenated castor oil. In a most preferred embodiment, the antiperspirant product comprises about 20-22 wt. % stearyl alcohol and 2.8 wt. % hydrogenated castor oil.

The antiperspirant products also may comprise a high refractive index (R.I.) hydrophobic compound. As used herein, the term “high refractive index” means a refractive index of no less than about 1.4. The high R.I. hydrophobic compound also facilitates the minimization and/or prevention of a white residue on the skin by masking the active antiperspirant salt that stays upon the skin upon evaporation of the carrier. Examples of high R.I. hydrophobic compounds for use in the antiperspirant products include PPG-14 butyl ether, C12-C15 alkyl benzoate, such as Finsolv TN® available from Innospec of the United Kingdom, and phenyl dimethicone. In a preferred embodiment, the antiperspirant product comprises PPG-14 butyl ether and, in a more preferred embodiment, the antiperspirant product comprises PPG-14 butyl ether in an amount of about 5 to about 15 wt. % of the total antiperspirant product. In a most preferred embodiment, the antiperspirant product comprises about 9.8 wt. % PPG-14 butyl ether.

In another exemplary embodiment, the antiperspirant product comprises one or more suspending agents that facilitate suspension of the active antiperspirant compound in the antiperspirant product, thereby minimizing the amount of active antiperspirant compound that settles out of the antiperspirant product during manufacture. Suitable suspending agents include clays and silicas. Examples of suitable silicas include fumed silicas and silica derivatives, such as silica dimethyl silylate. Suitable clays include bentonites, bectorites and colloidal magnesium aluminum silicates. In one exemplary embodiment, the antiperspirant product comprises about 0.5 to about 2.5 wt. % suspending agents. In another exemplary embodiment, the antiperspirant product comprises a mixture of silica and silica dimethyl silylate. In a preferred embodiment, the antiperspirant product comprises about 0.3 wt. % silica and about 1.4 wt. % silica dimethyl silylate.

In addition to the ingredients identified above, the antiperspirant product may comprise additives, such as those used in conventional antiperspirants. These additives include, but are not limited to, fragrances, including encapsulated fragrances, dyes, pigments, preservatives, antioxidants, moisturizers, and the like. These optional ingredients can be included in the antiperspirant product in an amount of 0 to about 20 wt. %. In a preferred embodiment, the antiperspirant product comprises about 1.9% myristyl myristate, which provides a conditioning effect to the skin.

The antiperspirant products, according to various embodiments, can be prepared by first combining the structurants and then melting them or, alternatively, melting each structurant and then mixing them. The remaining ingredients can be added to the melted structurants, either separately or as one or more premixtures, to form a liquid antiperspirant product, which is then poured into molds and permitted to cool at room temperature to form a solid stick antiperspirant product.

The following is an exemplary embodiment of an antiperspirant product contemplated herein, with each of the components set forth in weight percent of the antiperspirant product. The example is provided for illustration purposes only and is not meant to limit the various embodiments of the antiperspirant product in any way.

### Example

| Ingredient | Wt. % |
|---|---|
| Polydimethyl siloxane | 40.0 |
| Aluminum zirconium | 21.8 |
| pentachloride GLY | |
| Stearyl Alcohol | 22.0 |
| PPG-14 butyl ether | 9.8 |
| Hydrogenated castor oil | 2.8 |
| Myristyl Myristate | 1.9 |
| Silica | 0.3 |
| Silica dimethyl silylate | 1.4 |
| Total | 100.0 |

The antiperspirant product of the Example was prepared by combining the hydrogenated castor oil, stearyl alcohol, PPG-14 butyl ether, and myristyl myristate, melting the combination at 85° C., and then permitting the combination to cool to about 60-65° C. A premixture of aluminum zirconium pentachlorohydrex glycine, polydimethyl siloxane, silica, and silica dimethyl silylate was formed and then added to the cooled combination. The liquid was poured into a stick mold having a temperature of 52° C. and was permitted to cool to room temperature.

Accordingly, antiperspirant products that exhibit reduced white residue deposition over time compared to conventional antiperspirant products, in particular conventional antiperspirant products comprising cyclomethicone, and processes for making them have been provided. Specifically, the various embodiments of the antiperspirant products contemplated herein comprise an active antiperspirant compound and a carrier having a first volatility rate in an air ambient at about 50° C. and 1 atm after a first time period and a second volatility rate in an air ambient at about 50° C. and 1 atm after a second, longer time period, wherein the second volatility rate is less than the first volatility rate. In this regard, within the first time period after application of the antiperspirant product to the skin, the carrier evaporates quickly, leaving behind the active antiperspirant compound to plug sweat ducts of sweat-producing glands in the skin. After the first time period, the carrier evaporates at a slower volatility rate, thus remaining on the skin to help mask white residue.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant product comprising:
an active antiperspirant compound; and
a carrier having a first volatility rate in ambient air at about 50° C. and 1 atm. after a first time period and a second volatility rate in ambient air at about 50° C. and 1 atm. after a second time period, wherein the second volatility rate is less than the first volatility rate and the first time period is less than the second time period.

2. The antiperspirant product of claim 1, wherein the first volatility rate is in the range of about more than 7% weight loss after about 10 minutes and the second volatility rate is in the range of about less than 75% weight loss after about 100 minutes.

3. The antiperspirant product of claim 2, wherein the first volatility rate is in the range of about 10 to about 30% weight loss after about 10 minutes and the second volatility rate is in the range of about 20 to about 70% weight loss after about 100 minutes.

4. The antiperspirant product of claim 1, wherein the carrier comprises a material selected from the group consisting of polydimethyl siloxane, isoparaflin/alkane compounds, and mixtures thereof.

5. The antiperspirant product of claim 4, wherein the carrier comprises a mixture selected from the group consisting of 20/80 vol. %, 40/60 vol. %, and 50/50 vol. % cyclomethicone and polydimethyl siloxane, respectively.

6. The antiperspirant product of claim 5, wherein the cyclomethicone comprises cyclopentasiloxane or cyclotetrasiloxane.

7. The antiperspirant product of claim 4, wherein the carrier comprises a mixture selected from the group consisting of 20/80 vol. % and 40/60 vol. % cyclomethicone and isoparaffin/alkane, respectively.

8. The antiperspirant product of claim 7, wherein the cyclomethicone comprises cyclopentasiloxane or cyclotetrasiloxane.

9. The antiperspirant product of claim 4, wherein the carrier comprises a mixture selected from the group consisting of 20/80 vol. %, 40/60 vol. %, 50/50 vol. %, 60/40 vol. %, and 80/20 vol. % polydimethyl siloxane and an isoparaffin/alkane compound.

10. The antiperspirant product of claim 4, wherein an isoparaffin/alkane compound comprises C13-C16 isoparaffin/C12-C14 isoparaffin/C13-C15 alkane.

11. The antiperspirant product of claim 1, wherein the carrier is present in the antiperspirant product in an amount of about 30 to about 60 wt. %.

12. The antiperspirant product of claim 1, wherein the active antiperspirant compound is present in the antiperspirant product in an amount of about 8 to about 30 wt. %.

13. An antiperspirant product comprising:
an active antiperspirant compound; and
a. carrier comprising a material selected from the group consisting of polydimethyl siloxane, isoparaffin/alkane compounds, and combinations thereof.

14. The antiperspirant product of claim 13, wherein the carrier comprises a mixture selected from the group consisting of 20/80 vol. %, 40/60 vol. %, and 50/50 vol. %, cyclomethicone and polydimethyl siloxane, respectively.

15. The antiperspirant product of claim 14, wherein the cyclomethicone comprises cyclopentasiloxane or cyclotetrasiloxane.

16. The antiperspirant product of claim 13, wherein the carrier comprises a mixture selected from the group consisting of 20/80 vol. % 40/60 vol. %, 50/50 vol. %, 60/40 vol. %, and 80/20 vol. % polydimethyl siloxane and a isoparaffin/alkane compound, respectively.

17. The antiperspirant product of claim 13, wherein the carrier comprises a mixture selected from the group consisting of 20/80 vol. % and 40/60 vol. % cyclomethicone and isoparaffin/alkane, respectively.

18. The antiperspirant product of claim 17, wherein the cyclomethicone comprises cyclopentasiloxane or cyclotetrasiloxane.

19. The antiperspirant product of claim 13, wherein an isoparaffin/alkane compound comprises C13-C16 isoparaffin/C12-C14 isoparaffin/C13-C15 alkane.

20. A process for making an antiperspirant product, the process comprising the steps of:
providing a carrier having a first volatility rate after about 10 minutes in ambient air at about 50° C. and 1 atm and a second volatility rate after about 100 minutes in ambient air at about 50° C. and 1 atm, wherein the second volatility rate is less than the first volatility rate;
combining the carrier and an active antiperspirant compound at a first temperature to form a mixture; and
cooling the mixture to a lower second temperature.
